(19) United States
(12) Patent Application Publication
(10) Pub. No.: US 2007/0027110 A1
(43) Pub. Date: Feb. 1, 2007

(54) Double salts of (-)-hydroxycitric acid with an amine and a group II a metal and a process for preparing the same

(76) Inventors: Ganga R. Gokaraju, Andhra Pradesh (IN); Venkata Subbaraju Gottumukkala, Andhra Pradesh (IN); Venkateswarlu Somepalli, Andhra Pradesh (IN); Sridhar Pratha, Andhra Pradesh (IN)

Correspondence Address: RADER FISHMAN & GRAUER PLLC, LION BUILDING, 1233 20TH STREET N.W., SUITE 501, WASHINGTON, DC, 20036, US

(21) Appl. No.: 10/572,066

(22) PCT Filed: Feb. 17, 2004
(86) PCT No.: PCT/IN04/00045

§ 371 Date: Mar. 15, 2006

(51) Int. Cl. A61K 31/7008; A61K 31/555; C07F 3/00; C07F 3/06

(52) U.S. Cl. 514/62; 514/263.34; 514/471; 514/185; 514/494; 536/18.7; 544/226; 556/124

(57) ABSTRACT

This invention relates to novel double salt of (−)-hydroxycitric acid with an amine and zinc or a group II A metal. These compounds are stable and water soluble and are used as nutraceuticals, weight reducing agents and in beverages. The double salts have the following general formula (I) Wherein X is zinc or a metal belonging to group II A of the Periodic Table and Y is glucosamine, caffeine or a known anorexic amine residue.

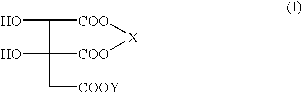

This invention relates to double salts of (−)-hydroxycitric acid with an amine and a group II A metal and a process for preparing the same.

### TECHNICAL FIELD

The (−)-hydroxycitric acid (HCA) is a naturally occurring fruit acid found in the rinds of the fruit of Garcinia cambogia (family: Guttiferae), a tree that is native to Southeast Asia and also found in other Garcinia species like Garcinia indica and Garcinia mangostana. The dried fruit rind of G. cambogia, also known as Malabar tamarind, is commonly used in Southeast Asia (particularly southern India) as a food preservative, flavoring agent and carminative. The primary mechanism of action of (−)-HCA appears to be related to act as a competitive inhibitor of the enzyme ATP-citrate lyase, which catalyzes the conversion of citrate and coenzyme A to oxaloacetate and acetyl coenzyme A (acetyl-CoA). Extensive experimental studies suggest that (−)-HCA suppresses the fatty acid synthesis, lipogenesis and food intake thus leading to weight reduction. In addition to suppression of fatty acid and fat synthesis, (−)-HCA is thought to suppress food intake via loss of appetite by stimulation of liver gluconeogenesis. Various researchers have evaluated HCA for its weight control properties, fat burning properties, lipid level lowering effect, appetite regulation, metabolic rate increase and other effects. A number of patents have been granted based on the above studies and various methods of extraction of HCA from the fruit. The isolation and chemical nature of (−)-hydroxycitric acid from Garcinia rind are described in the publication of Lewis, Y. S. et al, Phytochemistry, 4, 619-625 (1965). Moffett, et al., U.S. Pat. No. 5,656,314 (1997) described a process for the aqueous extraction of (−)-HCA from Garcinia rinds.

It has been found that the free acid form of (−)-hydroxycitric acid is unstable, forming lactone (FIG. 1) which generally do not possess the desired bioactivity and also the liquid form of HCA tends to be unstable during storage. Therefore, food preparations that incorporate the free acid in liquid form will not provide the full benefit of the HCA in the final preparation.

[image]

A number of patents have been granted for the preparation of (−)-hydroxycitric acid salts. Singh, et al., Biol. Memoirs, 21, 27-33 (1995), describes the preparation of calcium salt of HCA. The drawback of this salt is not very soluble in water. Majeed, et al., U.S. Pat. No. 5,783,603 (1998), disclosed the preparation of potassium salt of HCA; but it is hygroscopic and has strong pungent taste. Ganga Raju, G. PCT Publication No. WO 99/03464 (28 Jan., 1999) described the preparation of calcium and potassium or sodium double salts of HCA and its use as dietary supplements and food products to reduce body weight. Balasubramanyam, et al., U.S. Pat. No. 6,160,172 (2000) disclosed the preparation of similar double salts of HCA.

Calcium gives bones their strength, while magnesium helps them maintain their elasticity to prevent injury. The more calcium in the diet, the more magnesium that is needed. Calcium given alone can induce a magnesium deficiency. The most serious complications from a deficiency of magnesium are heart conditions such as irregular heartbeat and rapid heartbeat (Bariscode, M. et al., American Journal of Nutrition, 1996, 19, 296). The magnesium recommended in USA by the Daily Reference Intake (DRI) is 420 mg for male adults and 330 mg for female adults. So it is good to have good calcium and magnesium in our daily diet or supplement. Shrivatstava, et al, U.S. Pat. No. 6,221,901 (2001) disclosed the magnesium (−) hydroxycitrate as dietary nutritional supplement.

Zinc is an essential mineral that is found in almost every cell. It is needed for wound healing, sense of taste and smell, DNA synthesis and it supports normal growth and development during pregnancy, childhood and adolescence. The DRI's of zinc for adult male is 11 mg and for adult female is 8 mg.

Glucosamine (an amino saccharide) helps in strengthening the joint structure thereby improving mobility. So far four sources of glucosamine are reported namely glucosamine hydrochloride, glucosamine hydroiodide, glucosamine sulphate and N-acetyl glucosamine. Of these, glucosamine sulfate is the most preferred form of glucosamine and is widely used in the treatment of osteoarthritis and other acute and chronic forms of rheumatic and arthritic diseases. This salt in the market-place is labeled as ‘glucosamine sulfate’ or ‘stabilized glucosamine sulfate’, contains potassium or sodium chlorides. Ravi Gajanan, B et al, CA 2436925 (2002) and Schleck, et al, U.S. Pat. No. 5,902,801 (1999) described the preparation of glucosamine sulfate metal chloride by reacting glucosamine hydrochloride with metal sulfate.

Now we have invented, stable water soluble glucosamine (−)-hydroxycitric acid salts, which can be used both for arthritic diseases and to reduce body weight, since obese persons also suffer from arthritic diseases.

### DISCLOSURE OF THE INVENTION

The invention relates to double salts of (−)-hydroxycitric acid with an amine and zinc or a group II A metal having the following general formula 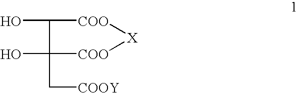
Wherein X is zinc or a metal selected from group II A of the Periodic Table and Y is glucosamine residue or caffeine or any known anorexic amine residue such as aminorex (4,5-dihydro-5-phenyl-2-oxazolamine), clobenzorex ((+)-N-[(2-chlorophenyl)methyl]-α-methylbenzeneethanamine), cyclexedrine (N,β-dimethylcycohexaneethanamine), diphemethoxidine (2-(diphenylmethyl)-1-piperidineethanol), fenbutrazate (α-ethylbenzeneacetic acid 2-(3-methyl-2-phenyl-4-morpholinyl)ethyl ester), fenproporex (3-[(1-methyl-2-phenylethyl)-amino]propanenitrile), furfurylmethylamphetamine (N-methyl-N-(1-methyl-2-phenylethyl)-2-furanmethanamine), levophacetoperane (α-phenyl-2-piperidinemethanol acetate), mefenorex (N-(3-chloropropyl)-α-methylbenzeneethanamine), metamfepramone (2-(dimethylamino)-1-phenylpropanone), norpseudoephedrine (threo-2-amino-1-hydroxy-1-phenylpropane), pentorex (α,α,β-trimethylbenzeneethanamine), picilorex (3-(4-chlorophenyl)-5-cyclopropyl-2-methylpyrrolidine), sibutramine (1-(4-chlorophenyl)-N,N-dimethyl-α-(2-methylpropyl)cyclobutanemethanamine) or phenylpropanolamine (2-amino-1-phenyl-1-propanol).

Preferably, this invention relates to double salts of (−)-hydroxycitric acid of the general formula 2 given below: 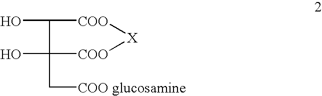
Wherein X is a group II A metal or zinc.

The new compounds according to this invention may be prepared by the following processes:
(a) by reacting (−)-hydroxycitric acid hereinafter referred as HCA with glucosamine and a compound containing zinc or group II A metal of the Periodic Table.
(b) by reacting calcium or magnesium or zinc and potassium/sodium double salts of HCA with glucosamine hydrochloride.

In the first process, stoichiometric equivalents of the reactants are mixed to obtain the desired double salts. Preferably, the reaction is initiated by the slow addition of glucosamine free base to an aqueous solution of HCA followed by the metal compound. Hydroxycitric acid may be obtained by a known process of extraction from the fruits of Garcinia species. The purified and enriched (−)—HCA may be obtained by converting water extract of the rind into insoluble calcium salt and converting back to HCA using phosphoric acid (Ganga Raju, G, PCT Publication No. WO 99/03464) or by passing the water extract of the fruit through ion exchange columns as described in U.S. Pat. No. 6,160,172.

Preferred metals selected are calcium, magnesium and zinc and these metal compounds are selected from their carbonates or hydroxides. Glucosamine base may be liberated from glucosamine hydrochloride by anionic exchange treatment.

The double salts prepared by this process may contain between 40 to 60% of HCA, 10-35% of glucosamine and 5-10% of the metal.

According to the second process of preparing the compounds of this invention, stoichiometric quantities of potassium/sodium and calcium double salts of HCA and glucosamine hydrochloride are reacted in aqueous methanol medium.

Compounds of this invention containing any amino substituents may be prepared by replacing glucosamine with the corresponding amino compounds.

Preferred compounds selected having amino residues are glucosamine, caffeine, norephedrine or sibutramine. The compounds according to this invention exhibit good water solubility, no taste or odour and are stable in comparison with the naturally occurring HCA.

Preferred embodiments of the processes of preparing the compounds of this invention are given in the following examples, which are in no way restrictive. These examples produce the compounds of this invention in high purity of more than 95%.

### EXAMPLE 1

Calcium, glucosamine double salt of (−)—HCA: To an aqueous garcinia solution (27 mL, 2.0 g, 1.0 equivalent) was added glucosamine free base solution (100 mL, 1.7 g, 1.0 equivalent) and stirred for 1 h. Then calcium hydroxide (708 mg, 1.0 equivalent) was added and after stirring for 2 h, charcoal (0.5 g) was added and stirred for 1 h. The solution was filtered through celite and was evaporated under reduced pressure to give glucosamine double salt of HCA as colourless powder (3.5 g).

The analytical characteristics of the glucosamine salt of HCA thus obtained are, (−)-HCA is 46.30%, lactone is 8.3%, glucosamine is 30.3% and calcium is 8.25%. This salt contains 1.01% of chlorides.

### EXAMPLE 2

Magnesium, glucosamine double salt of HCA: To an aqueous garcinia solution (27 mL, 2.0 g, 1.0 equivalent) was added glucosamine free base solution (100 mL, 1.7 g, 1.0 equivalent) and stirred for 1 h. Then magnesium carbonate (0.93 g, 1.0 equivalent) was added and after stirring for 2 h, charcoal (0.5 g) was added and stirred for 1 h. The solution was filtered through celite and stripped off water under reduced pressure to give magnesium, glucosamine double salt of HCA as colourless powder (3.0 g).

The analytical characteristics of the glucosamine salt of HCA obtained are, (−)—HCA: 49.80%, lactone: 6.3%, glucosamine: 31.3% and magnesium: 5.5%. This salt contains 0.5% of chlorides.

### EXAMPLE 3

Zinc, glucosamine double salt of HCA: To an aqueous garcinia solution (27 mL, 2.0 g, 1.0 equivalent) was added glucosamine free base solution (100 mL, 1.7 g, 1.0 equivalent) and stirred for 1 h. Then zinc carbonate (1.0 g, 1.0 equivalent) was added and after stirring for 2 h at rt, charcoal (0.5 g) was added and stirred for 1 h. The solution was filtered through celite and water was evaporated under reduced pressure to give zinc, glucosamine double salt of HCA as colourless powder (2.8 g).

The analytical characteristics of the glucosamine salt of HCA obtained are, (−)-HCA: 46.60%, lactone: 6.5%, glucosamine: 29.45% and zinc: 10.2%. This salt contains 0.8% of chlorides.

### EXAMPLE 4

Calcium, glucosamine double salt of HCA (KCl): A mixture of calcium, potassium salt of HCA (300 g, 1.0 molar equivalent, prepared by the procedure described in Ganga Raju, G. PCT Publication No. WO 99/03464) and glucosamine hydrochloride (209 g, 1.0 molar equivalent) in 95% aqueous methanol (2.0 L) was stirred at rt for 5 h. The solid was filtered and washed with the same solvent (250 mL). The solid mass was dried under vaccum at 60° C. for 5 h to give calcium, glucosamine double salt of HCA as colourless powder (495 g).

The analytical characteristics of the glucosamine salt of HCA thus obtained are, HCA: 40.10%, lactone: 0.37%, glucosamine: 36.81%, calcium: 6.96% and potassium: 8.48%. This salt contains 7.50% of chlorides.

### EXAMPLE 5

Calcium, norephedrine double salt of HCA: To an aqueous garcinia solution (27 mL, 2.0 g, 1.0 equivalent) was added norephedrine (1.4 g, 1.0 equivalent) and stirred for 1 h. Then calcium hydroxide (708 mg, 1.0 equivalent) was added and after stirring for 2 h, charcoal (0.5 g) was added and stirred for 1 h. The solution was filtered through celite and stripped off water under reduced pressure to give calcium, norephedrine double salt of HCA as colourless powder (2.7 g).

The analytical characteristics of the calcium, norephedrine double salt of (−)-HCA thus obtained are, HCA: 42.14%, lactone: 12.32%, norephedrine: 32.18% and calcium: 7.09%.

### EXAMPLE 6

Magnesium, norephedrine double salt of HCA: To an aqueous garcinia solution (27 mL, 2.0 g, 1.0 equivalent) was added norephedrine (1.4 g, 1.0 equivalent) and stirred for 1 h. Then magnesium carbonate (0.9 g, 1.0 equivalent) was added and after stirring for 2 h, charcoal (0.5 g) was added and stirred for 1 h. The solution was filtered through celite and water was evaporated under reduced pressure to give magnesium, norephedrine double salt of HCA as colourless powder (2.9 g).

The analytical characteristics of the magnesium, norephedrine double salt of HCA thus obtained are, HCA: 45.84%, lactone: 9.37%, norephedrine: 32.14% and magnesium: 5.62%.

### EXAMPLE 7

Zinc, norephedrine double salt of HCA: To an aqueous garcinia solution (27 mL, 2.0 g, 1.0 equivalent) was added norephedrine (1.4 g, 1.0 equivalent) and stirred for 1 h. Then zinc carbonate (1.0 g, 1.0 equivalent) was added and after stirring for 2 h, charcoal (0.5 g) was added and stirred for 1 h. The solution was filtered through celite and water was evaporated under reduced pressure to give zinc, norephedrine double salt of HCA as colourless powder (3.0 g).

The analytical characteristics of the zinc, norephedrine double salt of HCA thus obtained are, HCA: 42.83%, lactone: 10.35%, norephedrine: 30.47% and zinc: 10.2%.

### EXAMPLE 8

Magnesium, sibutramine double salt of HCA: To an aqueous garcinia solution (27 mL, 2.0 g, 1.0 equivalent) was added sibutramine (2.6 g, 1.0 equivalent) and stirred for 1 h. Then magnesium carbonate (0.9 g, 1.0 equivalent) was added and after stirring for 2 h, charcoal (0.5 g) was added and stirred for 1 h. The solution was filtered through celite and water was evaporated under reduced pressure to give magnesium, sibutramine double salt of HCA as colourless powder (3.5 g).

The analytical characteristics of the magnesium, sibutramine double salt of HCA thus obtained are, HCA: 38.58%, lactone: 10.16%, sibutramine: 40.0% and magnesium: 3.52%.

The new compounds are used in reducing body weight and as a cure for arthritis. These compounds may be used as a dietary and nutraceutical supplements particularly in beverages.

1. A double salt of (−)-hydroxycitric acid with an amine and zinc or a group II A metal having the following general formula. 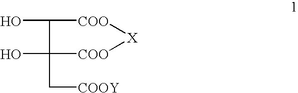
Wherein X is zinc or a metal belonging to group II A of the Periodic Table and Y is glucosamine or caffeine or a known anorexic amine residue.

2. The double salt as claimed in claim 1 wherein X is zinc or any group H A metal and Y is selected from glucosamine in free base form or glucosamine hydrochloride or other glucosamine salts.

3. The double salt as claimed in claim 1 wherein X is selected from zinc or any group II A metal and Y is selected from caffeine, norephedrine, sibutramine and oflaer anorexic amines.

4. The double salt as claimed in claim 1, wherein X is selected from Zn, Be, Mg, Ca, Sr, Ba or Ra.

5. The double salt as claimed in claim 1 wherein said anorexic amines are selected from aminorex, clobenzorex, cyclexedrine, diphemethoxidine, fenbutrazate, fenproporex, furfuryl methylamphetamine, levophacetoperane, mefenorex, metamfepramone, norpseudoephedrine, pentorex, picilorex, sibutramine or phenylpropanolamine.

6. A process for the preparation of a double salt of (−)-hydroxycitric acid with zinc or a group II A metal and a known amine of the general formula I comprising the steps of slowly adding the amine to an aqueous extract of hydroxycitric acid followed by the addition of a solution of group II A metal compound or a zinc compound.

7. The process as claimed in claim 6 wherein said double salt is recovered from the reaction mixture either by removing water under reduced pressure or by adding a water miscible solvent such as alcohols, acetone, acetonitrile, dioxan, tetrahydrofuran or mixtures thereof

8. A process for the preparation of double salt of (−)-hydroxycitric acid of the general formula I comprising the steps of displacing potassium or sodium from a calcium. votassium/sodium double salt of HCA by a known amine moiety in a known manner.

9. The process as claimed in claim 8, wherein said displacement is carried out in the presence of water or a mixture of water and water miscible solvents.

10. The process as claimed in claim 9, wherein said water miscible solvents are alcohols, acetone, acetonitrile, dioxan, totrahydrofuran or mixtures thereof.

11. The process as claimed in claim 9 wherein the water content of the water miscible solvent is in the range of 5 to 50% preferably in the range of 5 to 10%.

12. The process as claimed in claim 6 wherein said double salt is separated from the reaction mixture by filtration.

13. A process for preparing a double salt of (−)-hydroxycitric acid with anorexic amine compounds such as norephedrine or sibutramine and zinc or group II A metal compounds comprising the step of slowly adding 1.0 molar equivalent of norephedrine or sibutramine to an aqueous extract of hydroxycitric acid followed by adding 1.0 molar equivalent of zinc or group II A metal compound and recovering said double salt from the reaction mixture in a known manner.

14. The double salt of (−)-HCA with An ammie and zinc or a group II A metal according to claim 1 is more than 80%, preferably higher than 95%.

15. The double salt of (−)-HCA with glucosamine and zinc or a group II A metal composition according to claim 1 or 2 is approximately; HCA: 30-50%; lactone: 0.1-15%; glucosamine: 20-35%; calcium: 6-10% or magnesium: 4-8%; or zinc: 8-15% and chlorides: 0-2%.

16. The double salt of (−)-HCA with glucosamine and zinc or a group II A metal (KCl) composition according to claim 1 or 2 is approximately; HCA: 35-50%; lactone: 0.5-1.0%; glucosamine: 15-40%; calcium: 510%; potassium: 7-15% and chlorides: 2-8%.

17. The double salt of (−)-HCA with norephedrine and a group II A metal composition according to claim 1, comprises about 30 to about 40% of HCA; about 0.1 to about 10% of lactone; about 20 to about 35% of norephedrine and about 5 to about 10% of calcium.

18. The double salt of (−)-HCA with sibutramine and a group II A metal composition according to claim 1, comprises about 20 to about 35% of HCA; about 0.1 to about 10% of lactone: about 20 to about 45% of sibutramine and about 2 to about 8% of magnesium.

19. The double salt of (−)-HCA with an amine and zinc or a group II A metal are tasteless, odourless and highly soluble in water. These are suitable for nutraceutical or beverage applications.

20. A double salt (−)-hydroxycitric acid with an amine and zinc or a group II A metal substantially as herein described.

21. A process for preparing a double slat of (1)-hydroxycitric acid with an amine and zinc or group II A metal substantially as herein described and exemplified.

22. Use of double salt of (−)-hydroxycitric acid with an amine and zinc or a group II A metal for reducing obesity and for the treatment of arthritis.

23. Use of double salt of (−)-hydroxycitric acid with an amine and zinc or a group II A metal as dietary supplement, a nutraceufical and in beverages.
